(19) United States
(12) Patent Application Publication
(10) Pub. No.: US 2013/0310412 A1
(43) Pub. Date: Nov. 21, 2013

(54) Combinations of an Opioid/TLR4 Antagonist and a Direct-Acting Alpha-2 Adrenergic Agonist for Use in the Treatment of Pain

(71) Applicant: Annette Channa Toledano, North Miami, FL (US)

(72) Inventor: Annette Channa Toledano, North Miami, FL (US)

(21) Appl. No.: 13/799,287

(22) Filed: Mar. 13, 2013

Related U.S. Application Data
(63) Continuation-in-part of application No. 12/824,367, filed on Jun. 28, 2010.

(51) Int. Cl. A61K 31/485; A61K 31/4168

(52) U.S. Cl. CPC A61K 31/485; A61K 31/4168
USPC 514/282; 514/392

(57) ABSTRACT

Disclosed are compositions for treatment of pain comprising a first compound and a second compound, where the first compound is an opioid antagonist that treats pain by blocking Toll-like receptor 4 (TLR4) and the second compound is a direct-acting alpha-2 adrenergic agonist that enhances the pain treatment effect and abates adverse effects of the first compound, synergistic pharmaceutical compositions thereof, and their use in the treatment, prevention, and reversal of pain, particularly neuropathic pain.

### CROSS-REFERENCE TO RELATED APPLICATIONS

This application is a continuation-in-part of U.S. application Ser. No. 12/824,367 filed Jun. 28, 2010, which claims the benefit of U.S. Patent Provisional Application Ser. No. 61/343,489, entitled “Method for treating pain by a composition of naltrexone/clonidine at any dose combination” filed on Apr. 29, 2010 and Provisional Application Ser. No. 61/395,772 entitled “Compositions for treatment of pain, including but not exclusive to affect spinal pain (back pain), arthritic joint pain and migraine headache, pain of neuropathic and nociceptive origin” filed on May 17, 2010 the entire teachings of which are incorporated herein by reference.

### FIELD OF INVENTION

This invention relates to combinations of an opioid/TLR4 antagonist and an alpha-2 adrenergic receptor agonist, particularly those that exhibit a synergistic effect, for the treatment, prevention and reversal of pain.

### BACKGROUND

It is well established in medical literature that treatments currently available for pain have limitations. Opioid drugs cause tolerance, dependence and side effects sufficiently serious to prompt recent action by the FDA to further restrict the drugs. NSAIDs, taken for a prolonged periods of time, are known to cause gastro-intestinal bleeding as well as toxicity to the liver, kidneys and other organs. Newly approved treatments, like the calcium channel alpha-2-delta ligands gabapentin and pregabalin and the serotonin and norepinephrine reuptake inhibitors milnacipran and duloxetine, require high doses to show nominal effectiveness, have a high dropout rate and carry many side effects.

This invention is a novel approach for the treatment of pain syndromes associated with allodynia. It breaks the cycle of allodynia, or ‘memory pain’, as allodynia may be described in lay terms. Allodynia is a clinical feature of many painful conditions, such as neuropathies. The specific combination of drugs and the dosage needed to create that effect is the subject of this patent. The unifying theory of neuropathic pain is the understanding that TLR4 are activated endogenously and trigger a pro-inflammatory cascade. That cascade is interrupted and in most cases eliminated by treatment using the systemic administration of (1) an opioid/TLR4 antagonist, particularly naltrexone, or a pharmaceutically acceptable salt thereof and (2) a direct-acting alpha-2 adrenergic agonist, particularly clonidine, or a pharmaceutically acceptable salt thereof. Clonidine enhances the pain relief action and abates adverse effects of the opioid/TLR4 antagonist naltrexone. The specific synergistic dose range of the combination is herein clinically proven with high statistical significance and is superior to each and any existing therapy in the art of treating pain.

The instant invention discloses results of two double blind clinical trials conducted by the Applicant for subjects with back pain. One trial enrolled 24 subjects and the second enrolled 54 subject.

Results of the trials (see further discussion in Detailed Description of Invention) found that combining the opioid/TLR4 antagonist, naltrexone that blocks the opiate receptor TLR4, and the direct-acting alpha-2 adrenergic receptor agonist, clonidine, which enhances the pain treatment effect and abates the side effects of naltrexone. Using such combination in the treatment of back pain has greatly reduced the back pain, or in some cases, eliminated the pain entirely. In addition, in a dose finding study the combination of the opioid/TLR4 antagonist, naltrexone and the direct-acting alpha-2 adrenergic receptor agonist, clonidine, acted synergistically, whether administered separately, one right after the other, or administered in combination.

Various μ-opioid receptor ligands have been tested and were found to also possess action as agonists or antagonists of Toll-like receptor 4 (TLR4). Toll-like receptors, found in the glia, are a class of receptors that play a key role in the innate immune system. They recognize pathogen-associated molecular patterns (PAMPs) such as lipopolysaccharide (LPS) that are expressed on infectious agents, and mediate the production of cytokines necessary for the development of effective immunity. Opioid agonists such as morphine act as TLR4 agonists, while opioid antagonists such as naloxone and naltrexone were found to be TLR4 antagonists. Activation of TLR4 by opioid agonists such as morphine leads to downstream release of inflammatory modulators including TNF-α and interleukin-1. Constant low-level release of these modulators is thought to reduce the efficacy of opioid drug treatment with time and to be involved in both the development of tolerance to opioid analgesic drugs and in the emergence of side effects such as hyperalgesia and allodynia which can become problems following extended use of opioid drugs.

Accordingly, the instant invention relates to μ-opioid receptor ligands as ligands of TLR4 as well and contemplates that allodynia is caused by activation of TLR4. Blockage of TLR4 accordingly will eliminate allodynia.

The best known opioid receptor antagonists are naloxone and naltrexone. Naltrexone is an opioid receptor antagonist used primarily in the management of alcohol dependence and opioid dependence. A dose of 50-300 mg once daily is recommended for most patients. Naloxone is an opioid inverse agonist: it is a drug used to counter the effects of opiate overdose.

Low dose naltrexone describes the off label use of naltrexone at doses less than 10 mg per day for indications other than chemical dependency or intoxication.

It has been suggested in the literature that low dose naltrexone exerts the opposite effect of naltrexone in full dose. While the full dose naltrexone blocks the opiate system, the low dose naltrexone promotes the production of endorphins by the mechanism of up regulation caused by partial opiate receptor blockage. The beneficial effect of naltrexone was attributed to the increase in endorphins. The increase in endorphins may be the reason why it was found that low dose naltrexone has higher rates of the adverse reactions such as alertness, nervousness, anxiety and insomnia as a compared with the full dose. The full dose of naltrexone has a lower incidence of alertness, nervousness, anxiety, and insomnia because it lowers endorphins. The beneficial effect of low dose naltrexone can be explained by its action on the TLR4. The instant invention's clinical trial showed that the action of naltrexone on pain starts to occur with a low dose, and the effect increases with higher dose.

The package insert for full dose naltrexone reports single-digit incidence of nervousness, insomnia, and anxiety. In contrast, a study by Ploesser evaluating side effects of low dose naltrexone showed double digit incidence of nervousness, insomnia, and anxiety. (See Appendix A). This invention teaches using the direct-action alpha-2 adrenergic receptor agonist clonidine to abate the adverse effects caused by the low dose the opioid/TLR4 antagonist naltrexone. In the clinical trial of the instant invention, three treatment subjects out of 46 (6.5%) who received the study drug reported insomnia versus one subject (3.8%) in the placebo group which is a lower rate than what which was reported by Ploesser. The lower rate demonstrates the synergistic effect of the combination of this invention regarding the side effect profile.

Other opioid receptor antagonist used in clinical or scientific practice can also be used for the treatment of pain. They include but are not limited to the following: naloxone, nalmefene, norbinaltorphimine, nalorphine, methylnaltrexone, samidorphan, cyprodime, naltrindole, amentoflavone, naltriben, norbinaltorphimine, and the naltrexone metabolite 6-β-naltrexol.

Our understanding of pathological pain has primarily revolved around neuronal mechanisms. However, neighboring glia, were TLL4 reside, including astrocytes and microglia; have recently been recognized as powerful modulators of pain.

Studies show that TLRs can be activated not only by well-known ‘non-self’ molecular signals but also by endogenous signals (IL-1β, INFα, IL-6 and NO) produced during chronic neuropathic pain states. Fibronectin, an endogenous TLR4 ligand that is produced in response to tissue injury, leads to an up regulation of the purinoceptor P2X4, which is expressed exclusively on microglia.

Several opioid antagonist drugs were found to act as antagonists for TLR4, including naloxone and naltrexone. However it was found that not only the “normal” (−) enantiomers, but also the “unnatural” (+) enantiomers of these drugs acted as TLR4 antagonists. The unnatural enantiomers of the opioid antagonists, (+)-naltrexone and (+)-naloxone, dextro-naltrexone and dextro-naloxone, have been discovered to act as selective antagonists of TLR4. Since (+)-naloxone and (+)-naltrexone lack affinity for opioid receptors, they do not block the effects of opioid analgesic drugs, and so can be used to counteract the TLR4-mediated side effects of opioid agonists without affecting analgesia. (+)-Naloxone was also found to be neuroprotective, and both (+)-naloxone and (+)-naltrexone are effective in their own right at treating symptoms of neuropathic pain in animal models.

Clonidine is a sympatholytic medication, classified as a direct-acting α-2 adrenergic agonist. It is an imidazoline derivative. An alternative hypothesis that has been proposed is that clonidine acts centrally as an imidazoline-2 receptor agonist. The imidazoline-2 receptor is an allosteric binding site of monoamine oxidase and is involved in pain modulation and neuroprotection. Clonidine is used to treat medical conditions such as high blood pressure, ADHD, anxiety/panic disorder, and certain pain conditions. The therapeutic doses most commonly employed have ranged from 0.2 mg to 0.6 mg per day given in divided doses.

Clonidine enhances the pain treatment effect of naltrexone by agonism of the imidazoline-2 receptor, while its sympatholytic properties are the cause of the abatement of naltrexone's adverse reactions.

Although acute pain from an identifiable trauma is considered a symptom of tissue injury or disease, chronic and recurrent pain that persists for more than three months is itself a disease condition. Normally the pain pathway is activated to induce an adaptive and protective response that facilitates recuperation and helps to prevent further tissue damage. Once central sensitization begins, pain signaling is no longer the adaptive, protective mechanism that normal pain is and no longer serves for recuperation and wound healing.

Allodynia is a clinical feature of many painful conditions, such as back pain, chronic pain, neuropathic pain, diabetic neuropathic pain, trigeminal neuralgia pain, phantom limb pain, complex regional pain syndrome pain, acute herpetic pain, post herpetic pain, causalgia pain, idiopathic pain, inflammatory pain, cancer pain, postoperative pain, fibromyalgia pain, headache pain, migraine pain, allodynia pain, vulvodynia pain, interstitial cystitis pain, irritable bowel syndrome (IBS), arthritic joint pain and tendinitis. It becomes apparent that allodynia plays a role in every kind of pain.

The unifying theory of allodynia, or “memory pain”, is the understanding that TLR4 are activated endogenously and trigger a pro-inflammatory cascade. That cascade is interrupted by the opioid/TLR4 antagonist drugs as claimed by the instant invention. Additionally, TLR4 antagonism can play a role in improving nociceptive pain as well by affecting the allodynic component of nociceptive pain.

Based upon this, the instant invention first teaches the use of an opioid/TLR4 antagonist, particularly naltrexone for its antagonism of the TLR4 and blocking release of inflammatory modulators. Secondly, the invention teaches use of direct-acting alpha-2 adrenergic agonist, particularly clonidine, for its action on activation of the pain modulating receptor imidazoline. The instant invention teaches an additional benefit of clonidine abating the adverse effects of naltrexone. Therefore, the invention teaches that the combination has two dimensions of synergy: one of pain treatment effect and the other of abatement of side effects. The invention contemplates several forms of opioid antagonist selected from a group consisting of naltrexone, naloxone, nalmefene, norbinaltorphimine, nalorphine, methylnaltrexone, samidorphan, cyprodime, naltrindole, amentoflavone, naltriben, norbinaltorphimine, and metabolite 6-β-naltrexol and metabolites and pro drugs thereof, including all enantiomeric and epimeric forms as well as the appropriate mixtures thereof, or pharmaceutically acceptable salts or solvates of any thereof. Wherein, the direct-acting alpha-2 adrenergic agonist is selected from a group consisting of apraclonidine, brimonidine, clonidine, detomidine, dexmedetomidine, guanabenz, guanfacine, lofexidine, medetomidine, romifidine, tizanidine, tolonidine, xylazine and fadolmidine, or pharmaceutically acceptable salts or solvates of any thereof.

### SUMMARY OF INVENTION

The invention is a synergistic combination product comprising a first compound and a second compound, where the first compound is an opioid antagonist that treats pain by blocking receptor TLR4 and the second compound is a direct-acting alpha-2 adrenergic agonist that enhances the pain treatment effect and abates adverse reactions of the first compound, and the invention is a method for the treatment, prevention, and reversal of pain, particularly neuropathic pain.

### DESCRIPTION OF EMBODIMENTS

In one embodiment, the present invention discloses a method and a composition for treatment of pain in a mammal comprising a synergistic ratio of an opioid/TLR4 antagonist and a direct-acting alpha-2 adrenergic agonist, and pharmaceutically acceptable salts or solvate of any thereof.

Accordingly, in a preferred embodiment, the invention offers a method and a pharmaceutical composition of a therapeutically effective amount of an opioid antagonist and a direct-acting alpha-2 adrenergic agonist. The opioid/TLR4 antagonist is selected from a group consisting of naltrexone, norbinaltorphimine, nalmefene, naloxone, nalorphine, methylnaltrexone, samidorphan, cyprodime, naltrindole, amentoflavone, naltriben, norbinaltorphimine, 6-β-naltrexol and metabolites thereof, including all enantiomeric and epimeric forms as well as the appropriate mixtures thereof, as well as pro drugs or metabolites thereof or pharmaceutically acceptable salts or solvates of any thereof.

In another preferred embodiment, the invention offers a method and a pharmaceutical composition wherein the direct-acting alpha-2 adrenergic agonist is selected from a group consisting of clonidine, apraclonidine, brimonidine, detomidine, dexmedetomidine, guanabenz, guanfacine, lofexidine, medetomidine, romifidine, tizanidine, tolonidine, xylazine and fadolmidine or pharmaceutically acceptable salts or solvates of any thereof.

In a particular embodiment of a method and a pharmaceutical composition, the opioid antagonist/TLR4 is naltrexone as well as pro drugs and all enantiomeric and epimeric forms, specifically, (+)-naltrexone (dextro-naltrexone), as well as the appropriate mixtures thereof, or pharmaceutically acceptable salts or solvates of any thereof.

In a particular embodiment of a method and a pharmaceutical composition, the opioid antagonist/TLR4 is naltrexone in a sustained release formulation, as well as pro drugs thereof or any enantiomeric and epimeric forms thereof, as well as the appropriate mixtures thereof, or pharmaceutically acceptable salts or solvates of any thereof.

In a particular embodiment of a method and a pharmaceutical composition, the opioid antagonist/TLR4 is (+)-naltrexone (dextro-naltrexone), as well as pro drugs thereof or any enantiomeric and epimeric forms thereof, as well as the appropriate mixtures thereof, or pharmaceutically acceptable salts or solvates of any thereof.

In a particular embodiment of a method and a pharmaceutical composition, the direct-acting alpha-2 adrenergic agonist is clonidine, or a pharmaceutically acceptable salt or solvate thereof.

In a particular embodiment of a method and a pharmaceutical composition, the direct-acting alpha-2 adrenergic agonist is clonidine in a sustained release formulation, or a pharmaceutically acceptable salt or solvate thereof.

In a particular embodiment of a method and a pharmaceutical composition, the opioid/TLR4 antagonist is naltrexone or a pharmaceutically acceptable salt or solvate thereof, in a therapeutically effective amount and the direct-acting alpha-2 adrenergic agonist is clonidine, or a pharmaceutically acceptable salt or solvate thereof, in a therapeutically effective amount.

In a particular embodiment of a method and a pharmaceutical composition, naltrexone, or a pharmaceutically acceptable salt or solvate thereof, and clonidine, or a pharmaceutically acceptable salt or solvate thereof, are in a weight to weight combination range which corresponds to a synergistic combination range of the order of 90:1 to 22.5:1 parts by weight.

In a particular embodiment of a method and a pharmaceutical composition, the dose range of naltrexone, or a pharmaceutically acceptable salt or solvate thereof, is about 0.004 mg/kg-0.71 mg/kg.

In a particular embodiment of a method and a pharmaceutical composition, the dose range of clonidine, or a pharmaceutically acceptable salt or solvate thereof, is about 0.00018 mg/kg-0.0086 mg/kg per day.

In a particular embodiment of a method and a pharmaceutical composition, the human dose range of naltrexone, or a pharmaceutically acceptable salt or solvate thereof, is 0.25 mg-50 mg per day.

In a particular embodiment of a method and a pharmaceutical composition, the human dose range of naltrexone, or a pharmaceutically acceptable salt or solvate thereof, is 0.25 mg-25 mg per day.

In a particular embodiment of a method and a pharmaceutical composition, the human dose range of naltrexone, or a pharmaceutically acceptable salt or solvate thereof, is 0.25 mg-15 mg per day.

In a particular embodiment of a method and a pharmaceutical composition, the human the dose range of clonidine, or a pharmaceutically acceptable salt or solvate thereof, is 0.0125 mg-0.6 mg.

In a particular embodiment of a method and a pharmaceutical composition, the human the dose range of clonidine, or a pharmaceutically acceptable salt or solvate thereof, is 0.0125 mg-0.3 mg.

In a particular embodiment of a method and a pharmaceutical composition, the human dose range of naltrexone, or a pharmaceutically acceptable salt or solvate thereof, is 0.25 mg-50 mg per day, and the human the dose range of clonidine, or a pharmaceutically acceptable salt or solvate thereof, is 0.0125 mg-0.6 mg, wherein said composition is formulated into a single fixed combination dosage form.

In a particular embodiment of a method and a pharmaceutical composition, the composition is administered once, twice, three or four times through the day.

In a particular embodiment of a method and a pharmaceutical composition, the therapeutically effective dose of the pharmaceutical composition is administered systemically by such routes including but are not limited to mucosal, nasal, oral, parenteral, gastrointestinal, topical or sublingual routes.

In a particular embodiment of a method and a pharmaceutical composition, said combination is in a single dosage form, and said single dosage form is in the form of tablets, lozenges, troches, hard candies, liquid, powders, sprays, creams, salves and suppositories.

In a particular embodiment of a method and a pharmaceutical composition, the pharmaceutical composition is used for the treatment, prevention and reversal of neuropathic pain, back pain, chronic pain, diabetic neuropathic pain, trigeminal neuralgia pain, phantom limb pain, complex regional pain syndrome pain, acute herpetic pain, post herpetic pain, causalgia pain, idiopathic pain, inflammatory pain, cancer pain, postoperative pain, fibromyalgia pain, headache pain, migraine pain, allodynia pain, vulvodynia pain, interstitial cystitis pain, irritable bowel syndrome (IBS), arthritic joint pain and tendinitis.

In a particular embodiment of a method and a pharmaceutical composition, the pharmaceutical composition is used for the treatment, prevention and reversal of pain wherein said pain is back pain.

In a particular embodiment of a method and a pharmaceutical composition, the pharmaceutical composition is used for the treatment, prevention and reversal of pain wherein said pain is neuropathic pain.

In a particular embodiment of a method and a pharmaceutical composition, the pharmaceutical composition is used for the treatment, prevention and reversal of pain wherein said pain is migraine headache.

In a particular embodiment of a method and a pharmaceutical composition, the pharmaceutical composition is used for the treatment, prevention and reversal of pain wherein said pain is trigeminal neuralgia.

In a particular embodiment of a method and a pharmaceutical composition, the pharmaceutical composition is used for the treatment, prevention and reversal of pain wherein said pain is vulvodynia.

In a particular embodiment of a method and a pharmaceutical composition, the pharmaceutical composition is used for the treatment, prevention and reversal of pain wherein said pain is irritable bowel syndrome.

In a particular embodiment of a method and a pharmaceutical composition, the pharmaceutical composition is used for the treatment, prevention and reversal of pain wherein said pain is post herpetic neuralgia.

In a particular embodiment of a method and a pharmaceutical composition, the pharmaceutical composition is used for the treatment, prevention and reversal of pain wherein said pain is diabetic neuropathy.

In a particular embodiment of a method and a pharmaceutical composition, the pharmaceutical composition is used for the treatment, prevention and reversal of nociceptive pain with an allodynic component.

In a particular embodiment of a method and a pharmaceutical composition, the combination of naltrexone, or a pharmaceutically acceptable salt or solvate thereof, and clonidine, or a pharmaceutically acceptable salt solvate thereof may optionally be administered with one or more other pharmacologically active agents. Appropriate optional agents include: non-steroidal anti-inflammatory drugs (NSAIDs), e.g. aspirin, ibuprofen, naproxen, Naprosyn, Diclofenac, Ketoprofen, Tolmetin, Sulindac, Mefanamic acid, mMclofenamic acid, Diflunisal, Flufenisal, Piroxicam, Sudoxicam, Isoxicam, Celecoxib, Fofecoxib, Flosulide, Neloxicam, 6-methoxy-2-naphthylacetic acid, Nabumetone, Nimesulide, steroidal anti-inflammatory drugs, Tricyclic antidepressants (TCAs), selective serotonin reuptake inhibitors (SSRIs), serotonin-norepinephrine reuptake inhibitors (SNRIs), Anticonvulsants, muscle relaxants, drugs with NMDA antagonist properties, Tetrahydrocannabinol derivatives, Antitussive, Expectorants, Decongestants, or Antihistamines.

In a particular embodiment of a method and a pharmaceutical composition for non-human animal administration the term ‘pharmaceutical’ as used herein may be replaced by ‘veterinary’.

### DETAILED DESCRIPTION OF THE INVENTION

The present invention will now be described more fully hereinafter with reference to the accompanying drawings, in which preferred embodiments of the invention are shown. This invention may, however, be embodied in many different forms and should not be construed as limited to the embodiments set forth herein. Rather, these embodiments are provided so that this disclosure will be thorough and complete, and will fully convey the scope of the invention to those skilled in the art.

### Pharmaceutical Composition

Naltrexone and clonidine were evaluated alone and in combination on a human subject with the purpose of finding whether or not a combination of the two compounds offers synergistic advantage. Two aspects were evaluated for synergy: one aspect was pain treatment effect comparing the amounts used weight to weight, and the other aspect was an assessment of synergy of side effects.

The components of the combination were administered to a subject in the following manner: naltrexone 4.5 mg was given in the morning. The clonidine dose was divided into two doses, a dose of 0.1 mg in the morning and a dose of 0.2 mg at bedtime. The morning/daytime combination of naltrexone/clonidine 2.25 mg/0.025 mg respectively was given in the morning, and the night/bedtime combination of naltrexone/clonidine of 2.25 mg/0.05 mg and 2.25 mg/0.1 mg respectively was given at night. The pain treatment effect of naltrexone and clonidine was evaluated after one hour post-dose. Side effects were assessed over the next 24 hours.

To determine synergy, the amounts of naltrexone and clonidine administered alone were compared to the combination combined amounts. For proper weight to weight (W/W) comparison between naltrexone and clonidine an adjustment for the higher potency of clonidine needed to be made based on the dose of each compound given by itself, where the naltrexone dose was 4.5 mg and the clonidine dose was 0.3 mg. Clonidine is 15 times more potent than naltrexone (4.5/0.3=15). Naltrexone and clonidine were administered at fixed dose ratios of 90:1, 45:1 and 22.5:1 to a human subject afflicted with neuropathic back pain.

Table 1 illustrates the naltrexone/clonidine ratios that exhibit weight to weight (W/W) synergy in a human subject. The 90:1 combination represents a 2-fold lower dose of naltrexone and 4-fold lower dose of clonidine when administered alone. The 45:1 combination represents a 2-fold lower dose of naltrexone and 2-fold lower dose of clonidine when administered alone.

TABLE 1
Naltrexone/Clonidine Ratios
And Weight to Weight (W/W) Synergy

| | | | Clonidine | | Total dose | |
|---|---|---|---|---|---|---|
| | | | Potency | % | Naltrexone + | |
| | Naltrexone | Clonidine | Adjustment | reversal | Adjusted | |
| Ratio | mg | mg | (×15) | of pain | clonidine mg | Interaction |
| 4.5:0 | 4.50 | — | | 100 | 4.5 | — |
| 0:0.3 | — | 0.300 | 4.500 | 50 | 4.5 | — |
| 90:1 | 2.25 | 0.025 | 0.375 | 100 | 2.625 | synergy |
| 45:1 | 2.25 | 0.050 | 0.750 | 100 | 2.25 + 0.75 = 3.0 | synergy |
| 22.5:1 | 2.25 | 0.1 | 1.500 | 100 | 2.25 + 1.5 = 3.75 | synergy |

The 22.5:1 ratio represents a 2-fold lower dose of naltrexone and the same dose of clonidine when administered alone. The fixed dose ratio of 90:1, 45:1 and 22.5:1 demonstrated weight to weight synergy with neuropathic back pain completely blocked by the doses of 2.25 mg/0.025 mg, 2.25 mg/0.05 mg and 2.25 mg/0.1 mg of naltrexone/clonidine respectively.

Table 2 demonstrates the side effect synergy of the naltrexone/clonidine combination: alertness and anxiety from naltrexone are counteracted by the somnolence and calmness caused by clonidine. Naltrexone administered by itself at a 4.5 mg dosage carries a high incidence of alertness, insomnia and anxiety which deters compliance. Clonidine administered by itself at a 0.3 mg dosage at bedtime causes excessive somnolence that carries over through the next day.

TABLE 2
Synergy of Side Effects for Naltrexone/Clonidine

| | Naltrexone | | | |
|---|---|---|---|---|
| Ratio | mg | Clonidine mg | Interaction | Side effects |
| 4.5:0 | 4.5 | — | — | Alertness, |
| | | | | Insomnia, |
| | | | | anxiety |
| 0:0.3 | — | 0.3 | — | Excessive |
| | | | | Somnolence, |
| | | | | calmness |
| 90:1 | 2.25 | 0.025 | synergy | No side |
| | | | | effects |
| 45:1 | 2.25 | 0.05 | synergy | Possible |
| | | | | Mild |
| | | | | Somnolence |
| 22.5:1 | 2.25 | 0.1 | synergy | Somnolence |

The 90:1 ratio is a suitable synergistic ratio for morning/daytime contemplated administration. It embodies a lower adjusted total weight to weight dose of 2.625 mg compared to 4.5 mg of naltrexone and clonidine alone. Furthermore, it offers balance between the side effects: alertness and anxiety from naltrexone are counteracted by calmness from clonidine.

The 45:1 ratio is a suitable synergistic ratio for night/bedtime contemplated use. It embodies a lower adjusted total weight to weight dose of 2.35 mg compare to 4.5 mg of naltrexone and clonidine alone. This ratio provides additional sedation for more restorative night sleep.

The 22.5:1 ratio is a synergistic ratio for night/bedtime use as it provides additional sedative effect.

To summarize the naltrexone/clonidine synergistic effect, the invention teaches that the optimal contemplated naltrexone, or a pharmaceutically acceptable salt or solvate thereof, to clonidine, or a pharmaceutically acceptable salt or solvate thereof, combination dosage ratio range is between 90:1 and 22.5:1. This dosage ratio range exhibits synergy of weight to weight proportion and of side effect profile.

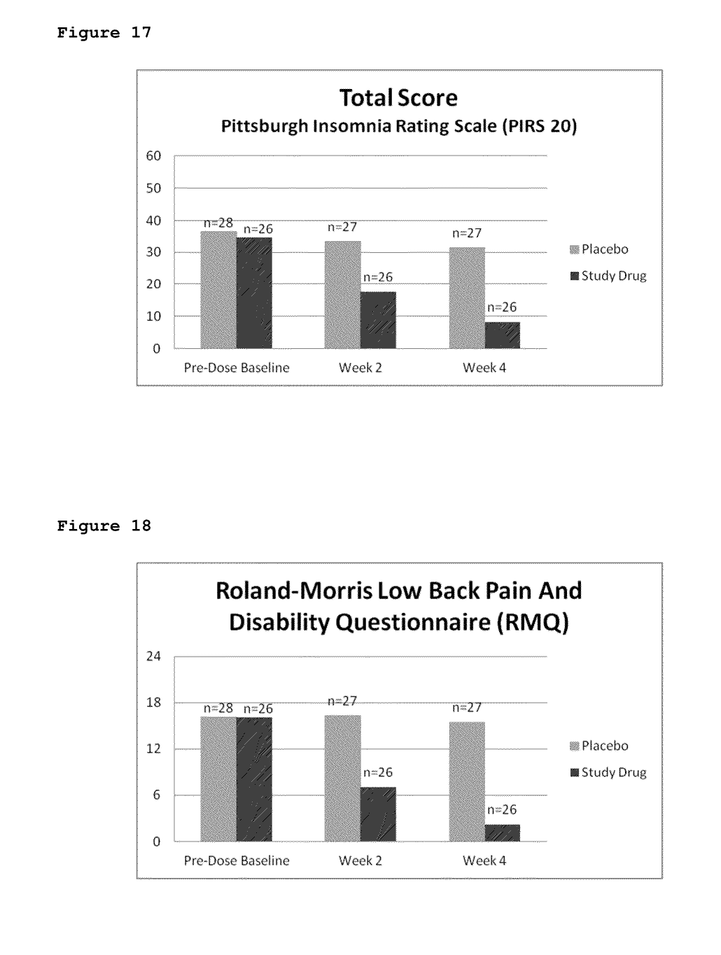

The instant invention clinical trial described below demonstrated statistically significant improvement of sleep compared to baseline as measured by the Pittsburgh Insomnia Rating Scale (PIRS 20). This suggests that the naltrexone side effects of alertness and insomnia were not only counteracted, but sleep was improved with the co-administration of the clonidine. See FIG. 17 Error! Reference source not found . . . .

### Examples

The examples disclosed in the instant application are case studies from the instant invention clinical trials. These examples are non-limiting and are merely representative of various aspects of the invention.

### An Example of Lower Back Pain Treatment Method

A 59 year old female presented with a 15 year history of lower back pain (LBP), leg weakness and pain. Additionally, she had a lifelong history of migraine headaches averaging two headache-days a week with an average intensity of 5 on a scale of 10, where 10 is intolerable pain. Prior to entering the double blind clinical trial, she had used duloxetine, topiramate, and non-steroidal anti-inflammatory drugs (NSAIDs) on a regular basis, all of which were discontinued upon entering the trial.

Prior to dosing, the subject could not stand on either leg for any length of time. Her baseline average back pain score the week before receiving study drug was 6.5 on a scale of 10, and her headache pain level was rated as 10. Eight minutes after the first dose combination of naltrexone/clonidine 2.25 mg/0.025 mg she reported that her headache had begun to resolve. After one hour her migraine pain had resolved to 0; her LBP had become 5 for a 60% improvement by her own report. After six hours, her LBP had fully resolved to a score of 0 out of 10.

The subject continued the combination of naltrexone/clonidine 2.25 mg/0.025 mg twice daily for 27 days during which time she reported no lower back pain or migraine pain. At the end of the 27-day treatment period she was able to stand separately on each leg for longer than 20 seconds.

During an 18-month post-study follow up period with no study medication, the subject was seen on six different occasions and maintained a self-reported long-term 80% improvement of her LBP and migraines compared to the period before receiving the study drug.

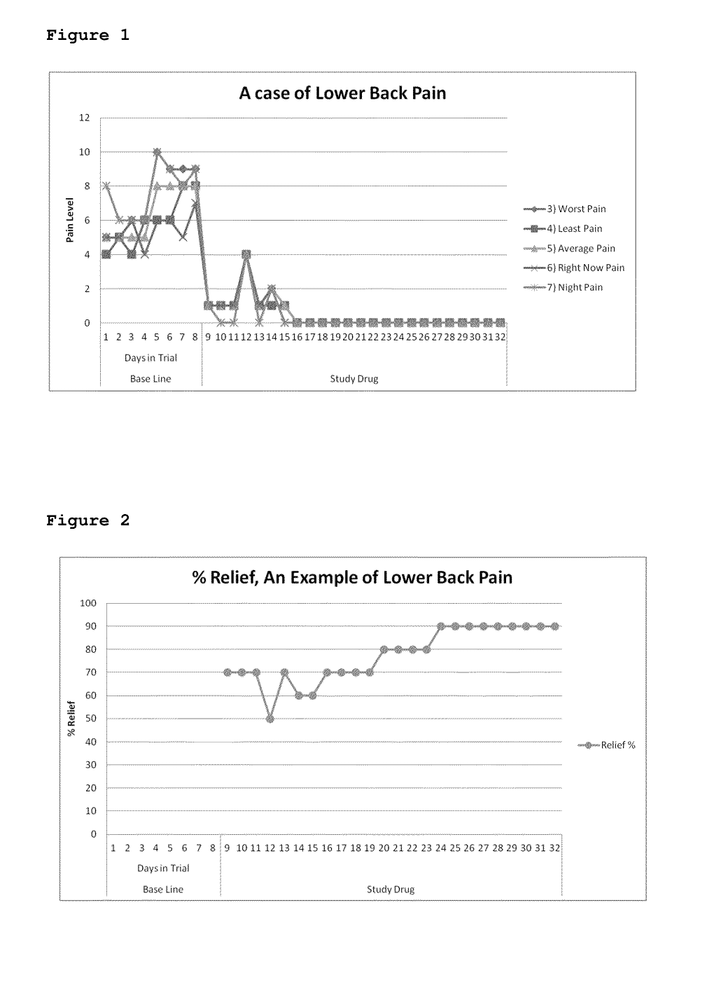

This case study shows that the combination of naltrexone/clonidine at the dose of 2.25 mg/0.025 mg twice daily is successful in treating acute migraine headache attack and chronic LBP. It also appears to have had a long-term prophylactic impact on migraine headaches. Table 3 tabulates the various pain intensity scores over time and percent relief for the example of lower back pain. The drawing in FIG. 1 is a graph of the various pain intensity scores over time in the case of lower back pain. The drawing in FIG. 2 is a graph of the percent relief reported by the case of lower back pain over time.

TABLE 3
an Example of Lower Back Pain
A case of Lower Back Pain

| | Base Line | | | | | | | | Study Drug | | | | | | | |
|---|---|---|---|---|---|---|---|---|---|---|---|---|---|---|---|---|
| | Days in Trial | | | | | | | | | | | | | | | |
| | 1 | 2 | 3 | 4 | 5 | 6 | 7 | 8 | 9 | 10 | 11 | 12 | 13 | 14 | 15 | 16 |
| 3) Worst | 5 | 5 | 6 | 6 | 10 | 9 | 9 | 9 | 1 | 1 | 1 | 4 | 1 | 2 | | 0 |
| Pain | | | | | | | | | | | | | | | | |
| 4) Least | 4 | 5 | 4 | 6 | 6 | 6 | 8 | 8 | 1 | 1 | 1 | 4 | 1 | 1 | 1 | 0 |
| Pain | | | | | | | | | | | | | | | | |
| 5) Average | 5 | 5 | 5 | 5 | 8 | 8 | 8 | 8 | 1 | 1 | 1 | 4 | 1 | 2 | 1 | 0 |
| Pain | | | | | | | | | | | | | | | | |
| 6) Right | 5 | 5 | 6 | 4 | 6 | 6 | 5 | 7 | 1 | 1 | 1 | 4 | 1 | 2 | 0 | 0 |
| Now Pain | | | | | | | | | | | | | | | | |
| 7) Night | 8 | 6 | 6 | 6 | 10 | 9 | 8 | 9 | 1 | 0 | 0 | 4 | 0 | 2 | 0 | 0 |
| Pain | | | | | | | | | | | | | | | | |
| Relief % | | | | | | | | | 70 | 70 | 70 | 50 | 70 | 60 | 60 | 70 |
| | Study Drug | | | | | | | | | | | | | | | |
| | 17 | 18 | 19 | 20 | 21 | 22 | 23 | 24 | 25 | 26 | 27 | 28 | 29 | 30 | 31 | 32 |
| 3) Worst | 0 | 0 | 0 | 0 | 0 | 0 | 0 | 0 | 0 | 0 | 0 | 0 | 0 | 0 | 0 | 0 |
| Pain | | | | | | | | | | | | | | | | |
| 4) Least | 0 | 0 | 0 | 0 | 0 | 0 | 0 | 0 | 0 | 0 | 0 | 0 | 0 | 0 | 0 | 0 |
| Pain | | | | | | | | | | | | | | | | |
| 5) Average | 0 | 0 | 0 | 0 | 0 | 0 | 0 | 0 | 0 | 0 | 0 | 0 | 0 | 0 | 0 | 0 |
| Pain | | | | | | | | | | | | | | | | |
| 6) Right | 0 | 0 | 0 | 0 | 0 | 0 | 0 | 0 | 0 | 0 | 0 | 0 | 0 | 0 | 0 | 0 |
| Now Pain | | | | | | | | | | | | | | | | |
| 7) Night | 0 | 0 | 0 | 0 | 0 | 0 | 0 | 0 | 0 | 0 | 0 | 0 | 0 | 0 | 0 | 0 |
| Pain | | | | | | | | | | | | | | | | |
| Relief % | 70 | 70 | 70 | 80 | 80 | 80 | 80 | 90 | 90 | 90 | 90 | 90 | 90 | 90 | 90 | 90 |

### An Example of Vulvodynia

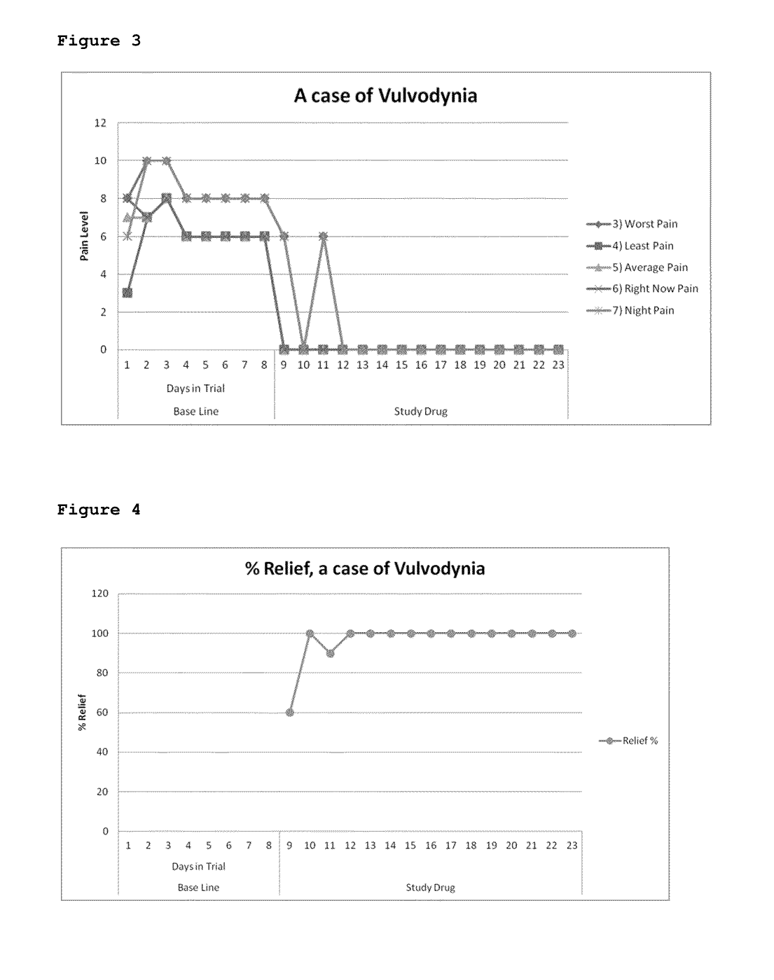

Vulvodynia is a chronic pain syndrome that affects the vulvar area and often occurs without an identifiable cause or visible pathology. A 55 year old female with a six year history of vulvodynia presented with constant pain and sensitivity in the vulva which interfered with her daily living and prevented her from having sexual intercourse. The subject entered open label study for three weeks. She improved dramatically after the first dose. After three days her pain level went from 9 to 0. After taking two capsules the first day, she noticed that she felt aggression, and so she reduced the dose to one capsule a day. One capsule still offered her full relief but without adverse events. Table 4 tabulates the various pain intensity scores over time and percent relief for the example of Vulvodynia. The drawing in FIG. 3 is a graph of the various pain intensity scores over time in the case of Vulvodynia pain. The drawing in FIG. 4 is a graph of the percent relief reported by the case of Vulvodynia pain over time.

TABLE 4
an Example of Vulvodynia
A case of Vulvodynia

| | Base Line | | | | | | | | Study Drug | | | | | |
|---|---|---|---|---|---|---|---|---|---|---|---|---|---|---|
| | Days in Trial | | | | | | | | | | | | | |
| | 1 | 2 | 3 | 4 | 5 | 6 | 7 | 8 | 9 | 10 | 11 | 12 | 13 | 14 |
| 3) Worst | 8 | 10 | 10 | 8 | 8 | 8 | 8 | 8 | 6 | 0 | 6 | 0 | 0 | 0 |
| Pain | | | | | | | | | | | | | | |
| 4) Least | 3 | 7 | 8 | 8 | 6 | 6 | 8 | 6 | 0 | 0 | 0 | 0 | 0 | 0 |
| Pain | | | | | | | | | | | | | | |
| 5) Average | 7 | 7 | 8 | 6 | 6 | 6 | 6 | 6 | 0 | 0 | 0 | 0 | 0 | 0 |
| Pain | | | | | | | | | | | | | | |
| 6) Right | 8 | 7 | 8 | 8 | 6 | 6 | 6 | 6 | 0 | 0 | 0 | 0 | 0 | 0 |
| Now Pain | | | | | | | | | | | | | | |
| 7) Night | 6 | 10 | 10 | 8 | 8 | 8 | 8 | 8 | 6 | 0 | 6 | 0 | 0 | 0 |
| Pain | | | | | | | | | | | | | | |
| Relief % | | | | | | | | | 60 | 100 | 90 | 100 | 100 | 100 |

| | Study Drug | | | | | | | | |
|---|---|---|---|---|---|---|---|---|---|
| | 15 | 16 | 17 | 18 | 19 | 20 | 21 | 22 | 23 |
| 3) Worst | 0 | 0 | 0 | 0 | 0 | 0 | 0 | 0 | 0 |
| Pain | | | | | | | | | |
| 4) Least | 0 | 0 | 0 | 0 | 0 | 0 | 0 | 0 | 0 |
| Pain | | | | | | | | | |
| 5) Average | 0 | 0 | 0 | 0 | 0 | 0 | 0 | 0 | 0 |
| Pain | | | | | | | | | |
| 6) Right | 0 | 0 | 0 | 0 | 0 | 0 | 0 | 0 | 0 |
| Now Pain | | | | | | | | | |
| 7) Night | 0 | 0 | 0 | 0 | 0 | 0 | 0 | 0 | 0 |
| Pain | | | | | | | | | |
| Relief % | 100 | 100 | 100 | 100 | 100 | 100 | 100 | 100 | 100 |

### An Example of Trigeminal Neuralgia

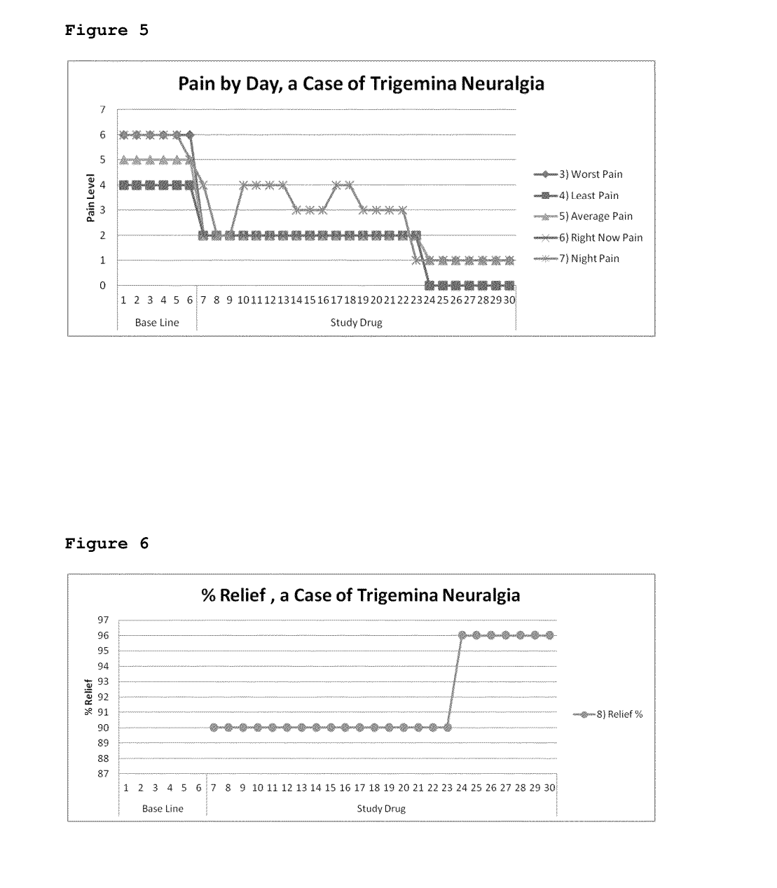

Trigeminal neuralgia, also known as suicide disease, is a neuropathic disorder characterized by episodes of intense pain in the face, originating from the trigeminal nerve. It has been described as among the most painful conditions known to mankind. This case study is of a 40 year old male with a three month history of trigeminal neuralgia. The patient had a gradual onset of pain in the right side of his face that started six weeks after a root canal. The pain involved the right check, upper jaw and the upper front teeth. The pain was constant and described as burning, stinging and aching. It was aggravated by eating, brushing teeth, flossing, chewing and talking. The subject's pain level on average was 5, but it would peak to 9. Ibuprofen brought the pain level from 9 to 7 for two hours, vicoden brought his pain level from 9 to 5, but relief lasted only three to four hours. The subject entered open label study for 3 weeks. After he was on the study drug at a dose of two capsules a day for one week his pain level fell to 2 and he reported being 90% better than before participating in the study. He stated that one dose had a lasting effect of up to 24 hours, unlike previous medications he had tried. Table 5 tabulates the various pain intensity scores over time and percent relief for the example of Trigeminal Neuralgia. The drawing in FIG. 5 is a graph of the various pain intensity scores over time in the case of Trigeminal Neuralgia pain. The drawing in FIG. 6 is a graph of the percent relief reported by the case of Trigeminal Neuralgia pain over time

TABLE 5
an Example of Trigeminal Neuralgia

| | Base Line | | | | | | Study Drug | | | | | | | | | |
|---|---|---|---|---|---|---|---|---|---|---|---|---|---|---|---|---|
| | 1 | 2 | 3 | 4 | 5 | 6 | 7 | 8 | 9 | 10 | 11 | 12 | 13 | 14 | 15 | 16 |
| 3) Worst | 6 | 6 | 6 | 6 | 6 | 6 | 2 | 2 | 2 | 2 | 2 | 2 | 2 | 2 | 2 | 2 |
| Pain | | | | | | | | | | | | | | | | |
| 4) Least | 4 | 4 | 4 | 4 | 4 | 4 | 2 | 2 | 2 | 2 | 2 | 2 | 2 | 2 | 2 | 2 |
| Pain | | | | | | | | | | | | | | | | |
| 5) Average | 5 | 5 | 5 | 5 | 5 | 5 | 2 | 2 | 2 | 2 | 2 | 2 | 2 | 2 | 2 | 2 |
| Pain | | | | | | | | | | | | | | | | |
| 6) Right | 4 | 4 | 4 | 4 | 4 | 4 | 2 | 2 | 2 | 2 | 2 | 2 | 2 | 2 | 2 | 2 |
| Now Pain | | | | | | | | | | | | | | | | |
| 7) Night | 6 | 6 | 6 | 6 | 6 | 5 | 4 | 2 | 2 | 4 | 4 | 4 | 4 | 3 | 3 | 3 |
| Pain | | | | | | | | | | | | | | | | |
| 8) Relief % | | | | | | | 90 | 90 | 90 | 90 | 90 | 90 | 90 | 90 | 90 | 90 |

| | Study Drug | | | | | | | | | | | | | |
|---|---|---|---|---|---|---|---|---|---|---|---|---|---|---|
| | 17 | 18 | 19 | 20 | 21 | 22 | 23 | 24 | 25 | 26 | 27 | 28 | 29 | 30 |
| 3) Worst | 2 | 2 | 2 | 2 | 2 | 2 | 2 | 1 | 1 | 1 | 1 | 1 | 1 | 1 |
| Pain | | | | | | | | | | | | | | |
| 4) Least | 2 | 2 | 2 | 2 | 2 | 2 | 2 | 0 | 0 | 0 | 0 | 0 | 0 | 0 |
| Pain | | | | | | | | | | | | | | |
| 5) Average | 2 | 2 | 2 | 2 | 2 | 2 | 2 | 1 | 1 | 1 | 1 | 1 | 1 | 1 |
| Pain | | | | | | | | | | | | | | |
| 6) Right | 2 | 2 | 2 | 2 | 2 | 2 | 2 | 0 | 0 | 0 | 0 | 0 | 0 | 0 |
| Now Pain | | | | | | | | | | | | | | |
| 7) Night | 4 | 4 | 3 | 3 | 3 | 3 | 1 | 1 | 1 | 1 | 1 | 1 | 1 | 1 |
| Pain | | | | | | | | | | | | | | |
| 8) Relief % | 90 | 90 | 90 | 90 | 90 | 90 | 90 | 96 | 96 | 96 | 96 | 96 | 96 | 96 |

### An Example of Irritable Bowel Syndrome (IBS)

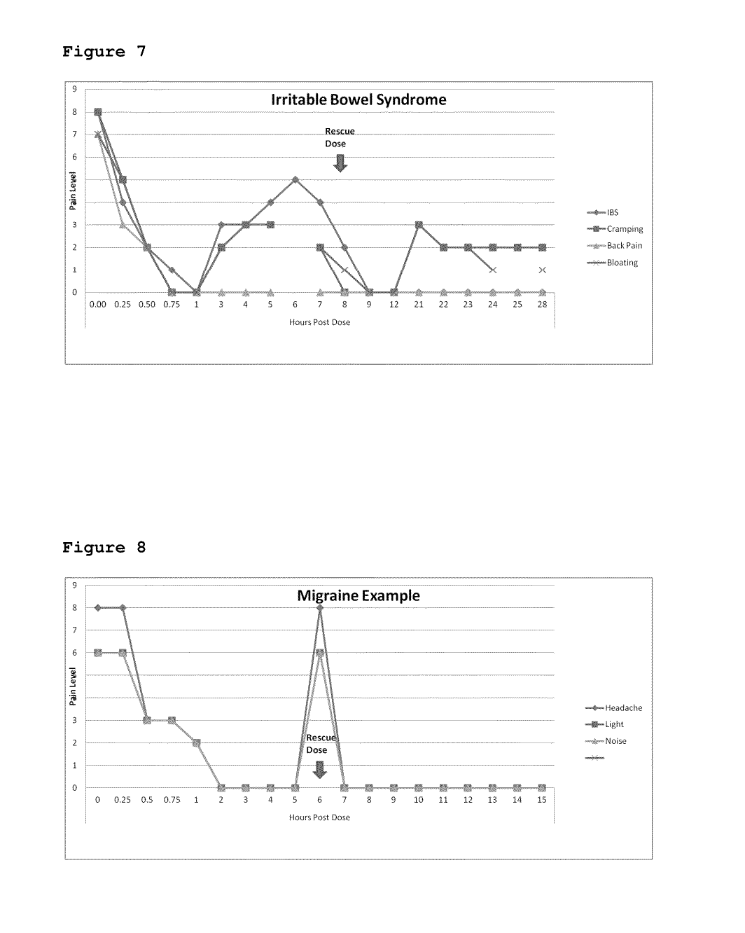

A 42 year old male had an eight year history of IBS averaging two attacks per year. His present flare-up had persisted for seven days with symptoms include left lower quadrant abdominal pain, cramping and bloating. Physical examination showed tenderness of the left lower quadrant and hyperactive bowel sounds. The subject reported a pain rating of 8. He enrolled in the open label phase of the trial with a baseline pain level of 8, lower back pain of 7. Seventy-five minutes after his initial dose, his pain, cramping, and lower back pain had become zero. Four hours later his pain recurred but was resolved after a second dose was taken. Table 6 tabulates the various IBS symptom intensity scores over time for the example of IBS. The drawing in FIG. 7 is a graph of the various IBS symptom intensity scores over time in the case of IBS.

TABLE 6
an Example of Irritable Bowel Syndrome
An Example of Irritable Bowel Syndrome

| | Hours Post Dose | | | | | | | | | | | | | | | | | | |
|---|---|---|---|---|---|---|---|---|---|---|---|---|---|---|---|---|---|---|---|
| | 0.00 | 0.25 | 0.50 | 0.75 | 1 | 3 | 4 | 5 | 6 | 7 | 8 | 9 | 12 | 21 | 22 | 23 | 24 | 25 | 28 |
| IBS | 8 | 4 | 2 | 1 | 0 | 3 | 3 | 4 | 5 | 4 | 2 | 0 | 0 | 0 | 0 | 0 | 0 | 0 | 0 |
| Cramping | 8 | 5 | 2 | 0 | 0 | 2 | 3 | 3 | | 2 | 0 | 0 | 0 | 3 | 2 | 2 | 2 | 2 | 2 |
| Back Pain | 7 | 3 | 2 | 0 | 0 | 0 | 0 | 0 | | 0 | 0 | 0 | 0 | 0 | 0 | 0 | 0 | 0 | 0 |
| Bloating | 7 | 5 | 2 | 0 | 0 | 2 | 3 | 3 | | 2 | 1 | 0 | 0 | 3 | 2 | 2 | 1 | | 1 |

### A Case of Acute Migraine

A 45 year old man presented with an acute migraine. His symptoms included pain level of 8 on a 0 to 10 point scale, with 10 being intolerable pain; nausea level of 5; and noise and light disturbance of 6. Seventy-five minutes after administration his nausea was at 3 while all his other symptoms went to zero, and he was able to look directly into the light. He was symptom-free for four hours, after which his headache and other symptoms recurred. He took two additional capsules of study drug and the symptoms resolved completely within one hour. Symptoms did not recur. Table 7 tabulates the various headache parameters intensity scores over time for the example of migraine. The drawing in FIG. 8 is a graph of the various headache parameters intensity scores over time in the case of migraine headache.

TABLE 7
an Example of Migraine
Migraine Example

| | Hours Post Dose | | | | | | | | | | | | | | | | | | |
|---|---|---|---|---|---|---|---|---|---|---|---|---|---|---|---|---|---|---|---|
| | 0 | 0.3 | 0.5 | 0.8 | 1 | 2 | 3 | 4 | 5 | 6 | 7 | 8 | 9 | 10 | 11 | 12 | 13 | 14 | 15 |
| Headache | 8 | 8 | 3 | 3 | 2 | 0 | 0 | 0 | 0 | 8 | 0 | 0 | 0 | 0 | 0 | 0 | 0 | 0 | 0 |
| Light | 6 | 6 | 3 | 3 | 2 | 0 | 0 | 0 | 0 | 6 | 0 | 0 | 0 | 0 | 0 | 0 | 0 | 0 | 0 |
| Noise | 6 | 6 | 3 | 3 | 2 | 0 | 0 | 0 | 0 | 6 | 0 | 0 | 0 | 0 | 0 | 0 | 0 | 0 | 0 |

### An Example of Leg Pain

A 61 year old woman with a ten year history of leg pain and weakness caused by L-5 radiculopathy was incapable of standing for longer than 10 minutes. She was not able to stand on one leg for any length of time on either side without losing balance immediately. The subject of this case had seen eleven spine surgeons, repeatedly told that surgery would not be beneficial for her pain. She tried oral medication such as NSAIDs, anticonvulsants, muscle relaxants, and anti-depressants (bupropion, duloxetine). Aside from some relief from NSAIDs, none of the other medications had any meaningful effect. She received spinal decompression on a DRX 9000 and obtained about 10% relief. An epidural steroid injection provided 5% relief for two weeks, three perispinal injections with botulinum toxin offered 10% relief for two months, and three perispinal Etanercept injections offered 10% relief for two months.

The subject in this case study has been on naltrexone/clonidine at the dose of 2.25 mg/0.025 mg twice daily for three years with improvement of up to 80% of her symptoms. Recently she increased the dose to 6 units which she prefers to take at night for the control of night pain and insomnia. With the current regiment her pain level stabilizes at a self-reported score of between 0-3 on a 10-point scale, and she has been able to stand on either leg for longer than 60 seconds. Without medication pain recurs within one to three days.

### Clinical Trial Excerpts

A placebo-controlled, double-blind, randomized, proof-of-concept clinical trial was conducted by the invention Applicant to determine the efficacy and safety of a low dose naltrexone and low dose clonidine combination for the treatment of symptoms of back pain. The studies were conducted under the regulatory oversight of the Food and Drug Administration (FDA) and the Independent Institutional Review Board (presently known as Shulman IRB). Fifty-four subjects were enrolled and treated for three weeks.

TABLE 8
Demographic Characteristics of Clinical Trial Subjects

| Characteristics | Study Drug | Placebo |
|---|---|---|
| Total N | 26 | 28 |
| Mean Age | 47.8 | 43.9 |
| Male:Female | 11:15 | 9:19 |
| Years of Lumbar Pain (n) | 8.75 yrs (n = 24) | 6.2 yrs (n = 20) |
| Years of Cervical Pain (n) | 6.2 yrs (n = 10) | 7.6 yrs (n = 16) |

The demographic characteristics of the subjects are summarized in table 8.

Table 9 shows 24 subjects out of 54 who entered the study for back pain had concomitant migraine or tension headache. This finding represents a higher incidence of headaches than found in the general population. Similarly, table 10 shows 26 out of 54 subjects had joint pain, most of which was associated with tendinitis rather than arthritic pain.

TABLE 9
Baseline Headache Pain Characteristics

| Characteristics | | Study Drug | Placebo |
|---|---|---|---|
| Migraine | N | 12 | 12 |
| | Ave. Duration | 16.8 yrs | 12.8 yrs |
| | Ave. Severity | 6 | 6.5 |
| Tension HA | N | 4 | 4 |
| | Ave. Duration | 26 yrs | 24 yrs |
| | Ave. Severity | 6.5 | 5 |

TABLE 10
Baseline Joint Pain Characteristics

| Characteristics | Study Drug | Placebo |
|---|---|---|
| N | 14 | 12 |
| Ave. Duration | 6.5 yrs | 3.3 yrs |
| Ave. Severity | 6.1 | 5.4 |

After a baseline period of 1 week, the subjects were randomized in 1:1 ratio study drug to placebo: 54 subjects received study drug or placebo for 3 weeks. The subjects took 1 capsule containing naltrexone/clonidine 2.25 mg/0.025 mg respectively or placebo twice daily and were instructed to increase the dose if the pain reduction effect was not sufficient. Subjects who did not respond had the option of continuing in an open label phase, receiving study drug for 3 week.

While in the study the subjects completed a daily pain questionnaire regarding back pain. Subjects who had concomitant headaches and/or joint pain recorded progression of those symptoms as well. The study subjects were evaluated in the office, in person four times during the course of the study and for subjects in open phase six times. Disability and sleep quality questionnaires along with safety data were collected during the office encounters.

Results

Table 11 presents the daily improvement of pain scores of study drug vs. placebo measured on a scale of 0-10, with 10 being most severe during the blinded study drug period.

TABLE 11
Daily Improvement of Pain Scores of Study Drug vs.
Placebo by Day

| | # of | # of | | | | | |
|---|---|---|---|---|---|---|---|
| | Subjects | Subjects | Study Drug | Placebo | Study Drug | | P |
| Days in | Receiving | Receiving | Average | Average | Minus | Standard | (Treatment > |
| Trial | Placebo | Study Drug | Pain Effect | Pain Effect | Placebo | Deviation | Placebo) |
| Day 2 | 27 | 26 | −2.61 | −0.56 | −2.05 | 0.24 | <.001 |
| Day 3 | 27 | 26 | −2.85 | −0.58 | −2.27 | 0.21 | <.001 |
| Day 4 | 27 | 26 | −3.15 | −0.58 | −2.57 | 0.20 | <.001 |
| Day 5 | 27 | 26 | −3.44 | −0.61 | −2.83 | 0.20 | <.001 |
| Day 6 | 26 | 26 | −3.54 | −0.62 | −2.92 | 0.20 | <.001 |
| Day 7 | 26 | 26 | −3.67 | −0.60 | −3.07 | 0.20 | <.001 |
| Day 8 | 26 | 26 | −3.83 | −0.59 | −3.24 | 0.20 | <.001 |
| Day 9 | 26 | 26 | −4.13 | −0.58 | −3.55 | 0.20 | <.001 |
| Day 10 | 26 | 26 | −4.29 | −0.58 | −3.71 | 0.20 | <.001 |
| Day 11 | 26 | 26 | −4.37 | −0.59 | −3.78 | 0.20 | <.001 |
| Day 12 | 26 | 26 | −4.54 | −0.58 | −3.96 | 0.20 | <.001 |
| Day 13 | 26 | 26 | −4.65 | −0.55 | −4.10 | 0.21 | <.001 |
| Day 14 | 26 | 26 | −4.73 | −0.54 | −4.19 | 0.21 | <.001 |
| Day 15 | 26 | 25 | −4.76 | −0.56 | −4.19 | 0.21 | <.001 |
| Day 16 | 26 | 24 | −4.88 | −0.60 | −4.28 | 0.21 | <.001 |
| Day 17 | 26 | 24 | −4.92 | −0.64 | −4.28 | 0.21 | <.001 |
| Day 18 | 26 | 24 | −4.93 | −0.68 | −4.25 | 0.21 | <.001 |
| Day 19 | 26 | 22 | −5.01 | −0.72 | −4.29 | 0.21 | <.001 |
| Day 20 | 22 | 20 | −5.01 | −0.77 | −4.25 | 0.22 | <.001 |
| Day 21 | 21 | 20 | −5.02 | −0.79 | −4.23 | 0.23 | <.001 |
| Day 22 | 18 | 15 | −5.05 | −0.83 | −4.22 | 0.25 | <.001 |
| Day 23 | 10 | 7 | −4.99 | −0.88 | −4.10 | 0.30 | <.001 |
| Day 24 | 5 | 5 | −4.93 | −0.91 | −4.02 | 0.33 | <.001 |
| Day 25 | 4 | 3 | −4.97 | −0.93 | −4.04 | 0.36 | <.001 |
| Day 26 | 2 | 2 | −5.03 | −0.93 | −4.10 | 0.39 | <.001 |
| Day 27 | 2 | 2 | −5.07 | −0.92 | −4.15 | 0.42 | <.001 |
| Day 28 | 0 | 2 | −5.10 | −0.92 | −4.17 | 0.45 | <.001 |
| Day 29 | 0 | 1 | −5.10 | −0.92 | −4.18 | 0.50 | <.001 |

Table 11 shows that back pain scores were significantly lower in the study drug subjects compared with placebo (P=0.001) from Day 2 onwards. The drug impact increases as time progressed showing a 5 point improvement.

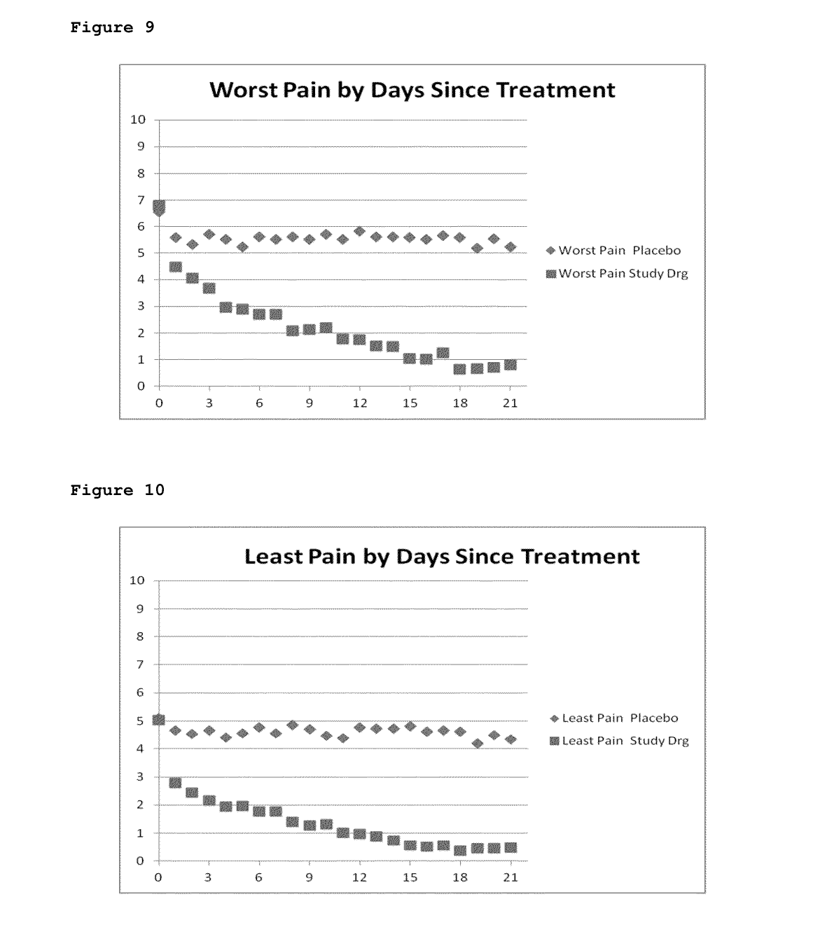

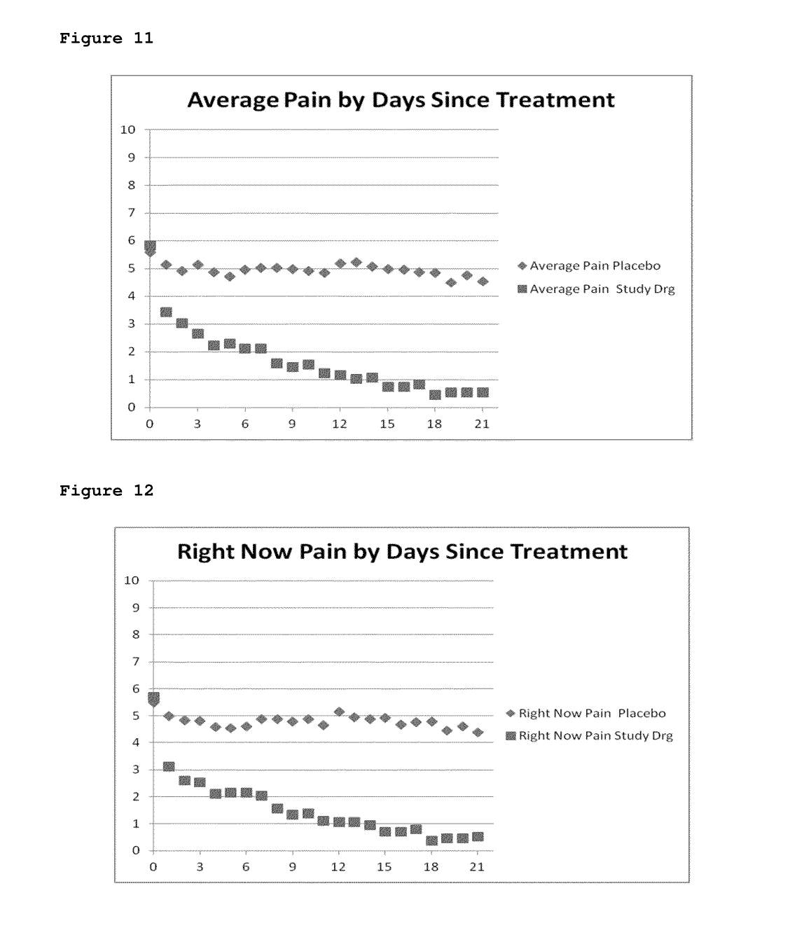

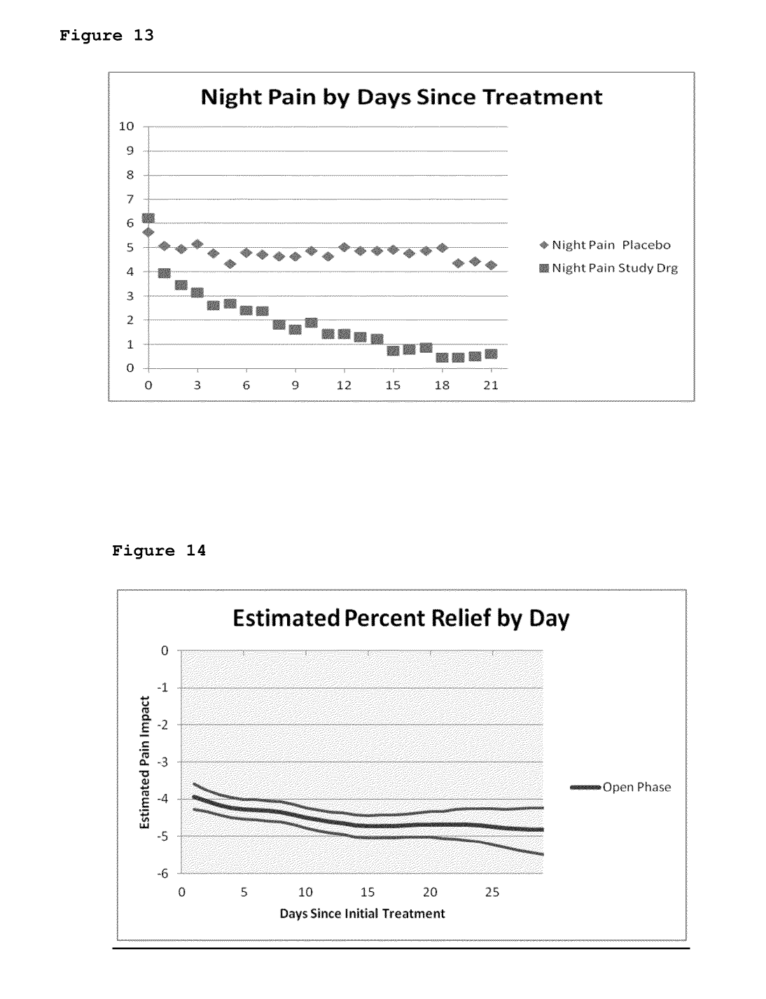

Pain intensity scores of study drug vs. placebo, as reported daily by the subjects for Worst Pain, Least Pain, Average Pain, Right-Now Pain, and Night Pain during the previous 24-hours are summarized in the following 5 tables and graphs. The data and graphs show consistent treatment impact on all pain intensity measures. Improvement begins starting the first day. The study drug impact increases as time progresses showing approximately a 5 point (on a scale of 0-10 with 10 being most severe) improvement compared to baseline by the end of the study. The Study Drug group exhibited resolution of pain. Table 12 tabulates the worst pain intensity scores over time for the study drug group versus placebo group in the clinical trial subjects. The drawing in FIG. 9 is a graph of the treatment impact on worst pain by day of study drug group versus placebo group in the clinical trial subjects. Table tabulates the least pain intensity scores over time for the study drug group versus placebo group in the clinical trial subjects. The drawing in FIG. 10 is a graph of the treatment impact on least pain by day of study drug group versus placebo group in the clinical trial subjects. Table 14 tabulates the average pain intensity scores over time for the study drug group versus placebo group in the clinical trial subjects. The drawing in FIG. 11 is a graph of the treatment impact on average pain by day of study drug group versus placebo group in the clinical trial subjects. Table 15 tabulates the right now pain intensity scores over time for the study drug group versus placebo group in the clinical trial subjects. The drawing in FIG. 12 is a graph of the treatment impact on right now pain by day of study drug group versus placebo group in the clinical trial subjects. Table 16 tabulates the night pain intensity scores over time for the study drug group versus placebo group in the clinical trial subjects. The drawing in FIG. 13 is a graph of the treatment impact on night pain by day of study drug group versus placebo group in the clinical trial subjects.

TABLE 12
Worst Pain Scores (Scale: 0-10) By Treatment Group

| | | Worst Pain Scores (Scale: 0-10) By Treatment Group | | | | | | | | | | |
|---|---|---|---|---|---|---|---|---|---|---|---|---|
| | | Study Day | | | | | | | | | | |
| | | B | 1 | 2 | 3 | 4 | 5 | 6 | 7 | 8 | 9 | 10 |
| Study | N | 26 | 26 | 26 | 26 | 26 | 26 | 26 | 26 | 26 | 26 | 26 |
| Drug | Ave. Score | 6.8 | 4.5 | 4.0 | 3.7 | 3.0 | 2.9 | 2.7 | 2.7 | 2.1 | 2.1 | 2.2 |
| Placebo | N | 27 | 27 | 27 | 27 | 27 | 26 | 26 | 26 | 26 | 26 | 26 |
| | Ave. Score | 6.5 | 5.6 | 5.3 | 5.7 | 5.5 | 5.2 | 5.6 | 5.5 | 5.6 | 5.5 | 5.7 |
| | | Worst Pain Scores (Scale: 0-10) By Treatment Group | | | | | | | | | | |
| | | Study Day | | | | | | | | | | |
| | | 11 | 12 | 13 | 14 | 15 | 16 | 17 | 18 | 19 | 20 | 21 |
| Study | N | 26 | 26 | 26 | 25 | 24 | 24 | 24 | 22 | 20 | 20 | 15 |
| Drug | Ave. Score | 1.8 | 1.7 | 1.5 | 1.5 | 1.0 | 1.0 | 1.3 | 0.6 | 0.7 | 0.7 | 0.8 |
| Placebo | N | 26 | 26 | 26 | 26 | 26 | 26 | 26 | 26 | 22 | 21 | 18 |
| | Ave. Score | 5.5 | 5.8 | 5.6 | 5.6 | 5.6 | 5.5 | 5.7 | 5.6 | 5.2 | 5.5 | 5.2 |

TABLE 13
Least Pain Scores (Scale: 0-10) By Treatment Group

| | | Least Pain Scores (Scale: 0-10) By Treatment Group | | | | | | | | | | |
|---|---|---|---|---|---|---|---|---|---|---|---|---|
| | | Study Day | | | | | | | | | | |
| | | B | 1 | 2 | 3 | 4 | 5 | 6 | 7 | 8 | 9 | 10 |
| Study | N | 26 | 26 | 26 | 26 | 26 | 26 | 26 | 26 | 26 | 26 | 26 |
| Drug | Ave. Score | 5 | 2.8 | 2 | 2.2 | 1.9 | 2 | 1.8 | 1.8 | 1.4 | 1.27 | 1.3 |
| Placebo | N | 27 | 27 | 27 | 27 | 27 | 26 | 26 | 26 | 26 | 26 | 26 |
| | Ave. Score | 5.1 | 4.7 | 5 | 4.7 | 4.4 | 4.5 | 4.8 | 4.5 | 4.8 | 4.69 | 4.5 |

| | | Least Pain Scores (Scale: 0-10) By Treatment Group | | | | | | | | | | |
|---|---|---|---|---|---|---|---|---|---|---|---|---|
| | | Study Day | | | | | | | | | | |
| | | 11 | 12 | 13 | 14 | 15 | 16 | 17 | 18 | 19 | 20 | 21 |
| Study | N | 26 | 26 | 26 | 25 | 24 | 24 | 24 | 22 | 20 | 20 | 15 |
| Drug | Ave. Score | 1 | 1 | 0.9 | 1 | 0.5 | 0.5 | 0.5 | 0 | 0.5 | 0.5 | 0.5 |
| Placebo | N | 26 | 26 | 26 | 26 | 26 | 26 | 26 | 26 | 22 | 21 | 18 |
| | Ave. Score | 4.4 | 4.8 | 4.7 | 5 | 4.8 | 4.6 | 4.7 | 5 | 4.2 | 4.5 | 4.3 |

TABLE 14
Average Pain Scores (Scale: 0-10) By Treatment Group

| | | Average Pain Scores (Scale: 0-10) By Treatment Group | | | | | | | | | | |
|---|---|---|---|---|---|---|---|---|---|---|---|---|
| | | Study Day | | | | | | | | | | |
| | | B | 1 | 2 | 3 | 4 | 5 | 6 | 7 | 8 | 9 | 10 |
| Study | N | 26 | 26 | 26 | 26 | 26 | 26 | 26 | 26 | 26 | 26 | 26 |
| Drug | Ave. Score | 5.8 | 3.4 | 3 | 2.7 | 2.2 | 2.3 | 2.1 | 2.1 | 1.6 | 1.46 | 1.5 |
| Placebo | N | 27 | 27 | 27 | 27 | 27 | 26 | 26 | 26 | 26 | 26 | 26 |
| | Ave. Score | 5.6 | 5.1 | 5 | 5.1 | 4.9 | 4.7 | 5 | 5 | 5 | 5 | 4.9 |

| | | Average Pain Scores (Scale: 0-10) By Treatment Group | | | | | | | | | | |
|---|---|---|---|---|---|---|---|---|---|---|---|---|
| | | Study Day | | | | | | | | | | |
| | | 11 | 12 | 13 | 14 | 15 | 16 | 17 | 18 | 19 | 20 | 21 |
| Study | N | 26 | 26 | 26 | 25 | 24 | 24 | 24 | 22 | 20 | 20 | 15 |
| Drug | Ave. Score | 1.2 | 1.2 | 1 | 1 | 0.8 | 0.8 | 0.8 | 0 | 0.6 | 0.6 | 0.5 |
| Placebo | N | 26 | 26 | 26 | 26 | 26 | 26 | 26 | 26 | 22 | 21 | 18 |
| | Ave. Score | 4.8 | 5.2 | 5.2 | 5 | 5 | 5 | 4.9 | 5 | 4.5 | 4.8 | 4.6 |

TABLE 15
Right Now Pain Scores (Scale: 0-10) By Treatment Group

| | | Right Now Pain Scores (Scale: 0-10) By Treatment Group | | | | | | | | | | |
|---|---|---|---|---|---|---|---|---|---|---|---|---|
| | | Study Day | | | | | | | | | | |
| | | B | 1 | 2 | 3 | 4 | 5 | 6 | 7 | 8 | 9 | 10 |
| Study | N | 26 | 26 | 26 | 26 | 26 | 26 | 26 | 26 | 26 | 26 | 26 |
| Drug | Ave. Score | 5.7 | 3.1 | 3 | 2.5 | 2.1 | 2.2 | 2.2 | 2 | 1.6 | 1.35 | 1.4 |
| Placebo | N | 27 | 27 | 27 | 27 | 27 | 26 | 26 | 26 | 26 | 26 | 26 |
| | Ave. Score | 5.5 | 5 | 5 | 4.8 | 4.6 | 4.5 | 4.6 | 4.9 | 4.9 | 4.81 | 4.9 |

| | | Right Now Pain Scores (Scale: 0-10) By Treatment Group | | | | | | | | | | |
|---|---|---|---|---|---|---|---|---|---|---|---|---|
| | | Study Day | | | | | | | | | | |
| | | 11 | 12 | 13 | 14 | 15 | 16 | 17 | 18 | 19 | 20 | 21 |
| Study | N | 26 | 26 | 26 | 25 | 24 | 24 | 24 | 22 | 20 | 20 | 15 |
| Drug | Ave. Score | 1.1 | 1.1 | 1.1 | 1 | 0.7 | 0.7 | 0.8 | 0 | 0.5 | 0.5 | 0.5 |
| Placebo | N | 26 | 26 | 26 | 26 | 26 | 26 | 26 | 26 | 22 | 21 | 18 |
| | Ave. Score | 4.7 | 5.2 | 5 | 5 | 4.9 | 4.7 | 4.8 | 5 | 4.5 | 4.6 | 4.4 |

TABLE 1
Night Pain Scores (Scale: 0-10) By Treatment Group

| | | Night Pain Scores (Scale: 0-10) By Treatment Group | | | | | | | | | | |
|---|---|---|---|---|---|---|---|---|---|---|---|---|
| | | Study Day | | | | | | | | | | |
| | | B | 1 | 2 | 3 | 4 | 5 | 6 | 7 | 8 | 9 | 10 |
| Study | N | 26 | 26 | 26 | 26 | 26 | 26 | 26 | 26 | 26 | 26 | 26 |
| Drug | Ave. Score | 6.2 | 4 | 3 | 3.2 | 2.6 | 2.7 | 2.4 | 2.4 | 1.8 | 1.62 | 1.9 |
| Placebo | N | 27 | 27 | 27 | 27 | 27 | 26 | 26 | 26 | 26 | 26 | 26 |
| | Ave. Score | 5.7 | 5.1 | 5 | 5.1 | 4.8 | 4.3 | 4.8 | 4.7 | 4.7 | 4.65 | 4.9 |

| | | Night Pain Scores (Scale: 0-10) By Treatment Group | | | | | | | | | | |
|---|---|---|---|---|---|---|---|---|---|---|---|---|
| | | Study Day | | | | | | | | | | |
| | | 11 | 12 | 13 | 14 | 15 | 16 | 17 | 18 | 19 | 20 | 21 |
| Study | N | 26 | 26 | 26 | 25 | 24 | 24 | 24 | 22 | 20 | 20 | 15 |
| Drug | Ave. Score | 1.4 | 1.4 | 1.3 | 1 | 0.8 | 0.8 | 0.9 | 0 | 0.5 | 0.5 | 0.6 |
| Placebo | N | 26 | 26 | 26 | 26 | 26 | 26 | 26 | 26 | 22 | 21 | 18 |
| | Ave. Score | 4.7 | 5 | 4.9 | 5 | 4.9 | 4.8 | 4.9 | 5 | 4.4 | 4.4 | 4.3 |

The Open Phase enrolled 22 subjects who had not responded to the initial treatment during the Study Drug Phase. Subjects received study drug twice daily for three to four weeks.

The Open Phase results are summarized in Table 17 and in FIG. 14. Table 17 tabulates the daily improvement of pain scores in the open phase and the drawing in FIG. 14 graphs the Open Phase Relief by Day. It shows that back pain symptoms were significantly lower in the open phase period compare with baseline measurement. Subjects who did not respond in the blinded study drug period, presumably the subjects who might have received the placebo in the blinded phase of the trial, were given the study drug containing low dose naltrexone and clonidine 2.25 mg/0.025 mg respectively for three weeks. Improvement begins starting the second day. The drug impact increases as time progress showing approximately a 5 point (on a scale of 0-10 with 10 being most severe) improvement compared to baseline by the end of the study.

TABLE 17
Daily Improvement of Pain Scores
Open Phase

| | | Treatment | Stan- | |
|---|---|---|---|---|
| | | with study | dard | P |
| | | drug compare | Devia- | (Treatment > |
| Days in Open Phase | Count | to base line | tion | Placebo) |
| Day 2 Of Open Phase | 22 | −3.94 | 0.18 | <.001 |
| Day 3 Of Open Phase | 22 | −4.05 | 0.15 | <.001 |
| Day 4 Of Open Phase | 21 | −4.16 | 0.15 | <.001 |
| Day 5 Of Open Phase | 21 | −4.23 | 0.15 | <.001 |
| Day 6 Of Open Phase | 22 | −4.27 | 0.15 | <.001 |
| Day 7 Of Open Phase | 21 | −4.29 | 0.15 | <.001 |
| Day 8 Of Open Phase | 21 | −4.32 | 0.15 | <.001 |
| Day 9 Of Open Phase | 20 | −4.35 | 0.15 | <.001 |
| Day 10 Of Open Phase | 20 | −4.42 | 0.15 | <.001 |
| Day 11 Of Open Phase | 19 | −4.50 | 0.15 | <.001 |
| Day 12 Of Open Phase | 19 | −4.57 | 0.15 | <.001 |
| Day 13 Of Open Phase | 17 | −4.62 | 0.16 | <.001 |
| Day 14 Of Open Phase | 16 | −4.65 | 0.15 | <.001 |
| Day 15 Of Open Phase | 15 | −4.71 | 0.16 | <.001 |
| Day 16 Of Open Phase | 14 | −4.73 | 0.17 | <.001 |
| Day 17 Of Open Phase | 14 | −4.73 | 0.17 | <.001 |
| Day 18 Of Open Phase | 10 | −4.72 | 0.17 | <.001 |
| Day 19 Of Open Phase | 18 | −4.71 | 0.17 | <.001 |
| Day 20 Of Open Phase | 18 | −4.69 | 0.18 | <.001 |
| Day 21 Of Open Phase | 9 | −4.68 | 0.19 | <.001 |
| Day 22 Of Open Phase | 8 | −4.69 | 0.19 | <.001 |
| Day 23 Of Open Phase | 3 | −4.68 | 0.21 | <.001 |
| Day 24 Of Open Phase | 3 | −4.69 | 0.22 | <.001 |
| Day 25 Of Open Phase | 3 | −4.71 | 0.23 | <.001 |
| Day 26 Of Open Phase | 2 | −4.74 | 0.25 | <.001 |
| Day 27 Of Open Phase | 1 | −4.78 | 0.27 | <.001 |
| Day 28 Of Open Phase | 1 | −4.80 | 0.29 | <.001 |
| Day 29 Of Open Phase | 1 | −4.82 | 0.30 | <.001 |

FIG. 14 shows an average 4.5 point drop in pain score during the Open Phase.

The instant invention clinical trial found that the opioid/TLR4 antagonist naltrexone, in combination with the alpha two adrenergic receptor agonist clonidine, treated and reversed chronic pain conditions, including chronic back pain, chronic headaches, including migraines and chronic joint pain. Additionally, the study drug reversed long standing trigeminal neuralgia, tactile allodynia, vulvodynia and IBS.

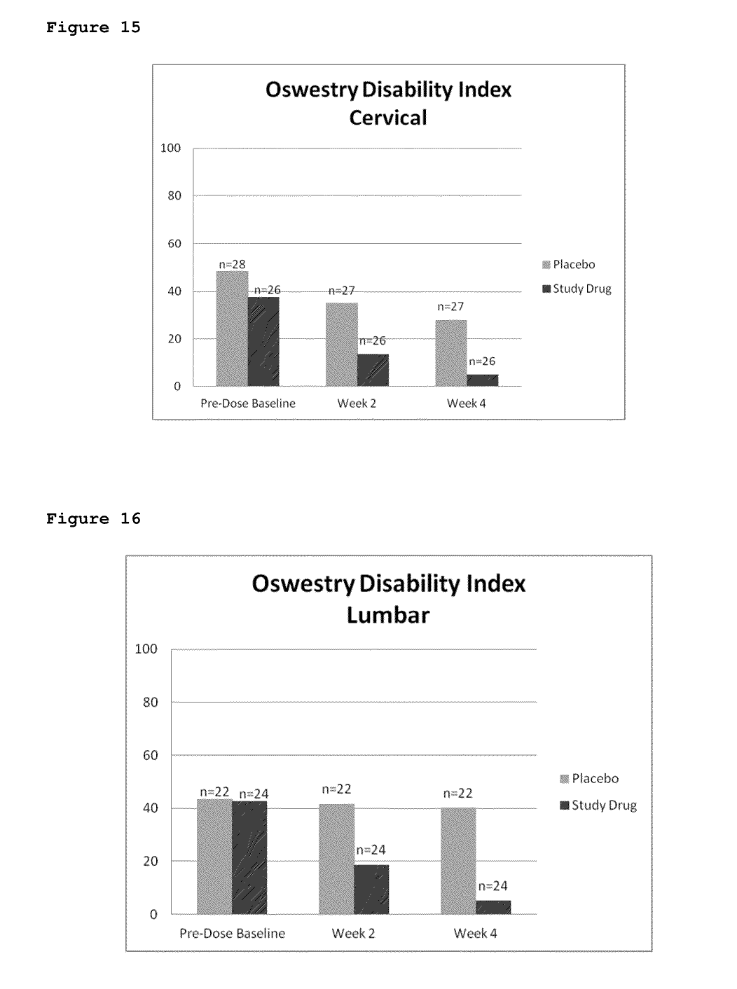

The Oswestry Disability Index is considered the gold standard for assessing the disability level of back pain for those, who suffer back pain, to assess their disability level. The drawing in FIG. 15 shows the change in the Oswestry Disability Index of the cervical pain over time in the study drug group versus the placebo group. The drawing in FIG. 16 shows the change in the Oswestry Disability Index of the lumbar pain over time in the study drug group versus the placebo group.

The Pittsburgh Insomnia Rating Scale (PIRS) was developed by the University of Pittsburgh's Western Psychiatric Institute and Clinic to assess insomnia. The Total Score is a sum of all nineteen responses to questions on the questionnaire with possible values of zero to sixty. Higher scores indicate greater degree of insomnia. The drawing in FIG. 17 shows the change in the Pittsburgh Insomnia Rating Scale over time in the study drug group versus the placebo group. The Study Drug treatment group showed statistically significant improvement over the placebo group in PIRS 20 Total Score at Week 2 and at Week 4.

The drawing in FIG. 18 graphs average the Roland-Morris Low Back Pain and Disability Questionnaire (RMQ) scores for the Study Drug Group and Placebo at Baseline, Week 2, and Week 4. The Roland-Morris Questionnaire is a self-administered disability measure in which greater levels of disability are reflected by higher numbers on a 24-point scale. The RMQ has been shown to yield reliable measurements, which are valid for inferring the level of disability, and to be sensitive to change over time for groups of patients with lower back pain.

From the clinical trial it is concluded that the opioid/TLR4 antagonist naltrexone, in combination with the alpha-2 adrenergic receptor agonist clonidine, treated and reversed the chronic pain conditions chronic back pain, chronic headaches including migraines and chronic joint pain. Additionally, the study drug reversed long standing trigeminal neuralgia, vulvodynia and irritable bowel syndrome.

### CITATION LIST

Bowling, Allen C. “Low-dose naltrexone (LDN) The “411” on LDN”. National Multiple Sclerosis Society. Retrieved 6 Jul. 2011.
Breivik H, Borchgrevink P C, Allen S M, Rosseland L A, Romundstad L, Hals E K, Kvarstein G, Stubhaug A. Assessment of pain. Br J. Anaesth. 2008; 101(1):17-24.doi:10.1093/bja/aen103. PMID 18487245.
Eur J Neurosci TNFα Levels and Macrophages Expression Reflect an Inflammatory Potential of Trigeminal Ganglia in a Mouse Model of Familial Hemiplegic Migraine Nat Rev Neurosci. 2009 January; 10(1): 23-36.
Hutchinson M R, Coats B D, Lewis S S, Zhang Y, Sprunger D B, Rezvani N, Baker E M, Jekich B M, Wieseler J L, Somogyi A A, Martin D, Poole S, Judd C M, Maier S F, Watkins L R (November 2008). “Proinflammatory cytokines oppose opioid induced acute and chronic analgesia”. Brain, Behavior, and Immunity 22 (8): 1178-89. doi:10.1016/j.bbi.2008.05.004. PMC 2783238.PMID 18599265.
Hutchinson M R, Lewis S S, Coats B D, Rezvani N, Zhang Y, Wieseler J L, Somogyi A A, Yin H, Maier S F, Rice K C, Watkins L R (May 2010). “Possible involvement of Toll-Like Receptor 4/MD-2 activity of opioid inactive isomers causes spinal proinflammation and related behavioral consequences”. Neuroscience 167 (3): 880-93. doi:10.1016/j.neuroscience.2010.02.011.PMC 2854318. PMID 20178837.
Hutchinson M R, et al. Opioid-induced glial activation: mechanisms of activation and implications for opioid analgesia, dependence and reward. Sci. World J. 2007; 7:98-111.
Hutchinson M R, et al. Non-stereoselective reversal of neuropathic pain by naloxone and naltrexone: involvement of toll-like receptor 4 (TLR4) Eur. J. Neurosci. 2008; 28:20-.
Hutchinson M R, Lewis S S, Coats B D, Rezvani N, Zhang Y, Wieseler J L, Somogyi A A, Yin H, Maier S F, Rice K C, Watkins L R (May 2010). “Possible involvement of Toll-Like Receptor 4/MD-2 activity of opioid inactive isomers causes spinal proinflammation and related behavioral consequences”. Neuroscience 167 (3): 880-93.doi:10.1016/j.neuroscience.2010.02.011. PMC 2854318. PMID 20178837.
Hutchinson M R, et al. Proinflammatory cytokines oppose opioid-induced acute and chronic analgesia. Brain Behav. Immun. 2008 Jul. 1.
Hutchinson M R, et al. Minocycline supresses morphine-induced respiratory depression, supresses morphine-induced reward, and enhances systemic morphine-induced analgesia. Brain Behav. Immun. 2008 Jul. 31.
Johnston I N, et al. A role for pro-inflammatory cytokines and fractalkine in analgesia, tolerance and subsequent pain facilitation induced by chronic intrathecal morphine. 2004; 24:7353-7365.
Komatsu T, Sakurada S, Katsuyama S, Sanai K, Sakurada T (2009). “Mechanism of allodynia evoked by intrathecal morphine-3-glucuronide in mice”. International Review of Neurobiology 85: 207-19. doi:10.1016/50074-7742(09)85016-2. PMID 19607972.
Lewis S S, Hutchinson M R, Rezvani N, Loram L C, Zhang Y, Maier S F, Rice K C, Watkins L R (January 2010). “Evidence that intrathecal morphine-3-glucuronide may cause pain enhancement via toll-like receptor 4/MD-2 and interleukin-1β”. Neuroscience 165 (2): 569-83. doi:10.1016/j.neuroscience.2009.10.011. PMC 2795035. PMID 19833175.
Mannelli P, Gottheil E, Van Bockstaele E J (2006). “Antagonist treatment of opioid withdrawal translational low dose approach”. J Addict Dis 25 (2): 1-8.doi:10.1300/J069v25n02_{—}01. PMID 16785213.
Milligan, E. D., and Watkins, L. R., Pathological and protective roles of glia in chronic pain, Nature Neuroscience Reviews, 10 (2009) 23-36.
Nasu-Tada K, Koizumi S, Tsuda M, Kunifusa E, Inoue K. Possible involvement of increase in spinal fibronectin following peripheral nerve injury in upregulation of microglial P2×4, a key molecule for mechanical allodynia. Glia. 2006; 53:769-775.
Ngian G S, Guymer E K, Littlejohn G O (February 2011). “The use of opioids in fibromyalgia” (PDF). Int J Rheum Dis 14 (1): 6-11. doi:10.1111/j.1756-185X.2010.01567.x. PMID 21303476.
Novella, Steven. “Low Dose Naltrexone—Bogus or Cutting Edge Science?”. Retrieved 5 Jul. 2011.
Obata K, et al. Toll-like receptor 3 contributes to spinal glial activation and tactile allodynia after nerve injury. J. Neurochem. 2008; 105:2249-2259.
Romero-Sandoval E A, Horvath R J, Deleo J A. Neuroimmune interactions and pain: focus on glial modulating targets. Curr. Opin. Investig. Drugs. 2008; 9:726-734.
Ploesser J, Weinstock L B, Thomas E. Low Dose Naltrexone: Side Effects and Efficacy in Gastrointestinal Disorders.
Tanga F Y, Nutile-McMenemy N, DeLeo J A. The CNS role of Toll-like receptor 4 in innate neuroimmunity and painful neuropathy. Proc. Natl. Acad. Sci. USA. 2005; 102:5856-5861.
Turk D C, Dworkin R H. What should be the core outcomes in chronic pain clinical trials?. Arthritis Res. Ther. 2004; 6(4):151-4. doi:10.1186/ar1196. PMID 15225358.
Watkins L R, Hutchinson M R, Rice K C, Maier S F (November 2009). “The “Toll” of Opioid-Induced Glial Activation: Improving the Clinical Efficacy of Opioids by Targeting Glia”. Trends in Pharmacological Sciences 30 (11): 581-91. doi:10.1016/j.tips.2009.08.002. PMC 2783351. PMID 19762094.
Webster L R (August 2007). “Oxytrex: an oxycodone and ultra-low-dose naltrexone formulation”. Expert Opin Investig Drugs 16 (8): 1277-83.doi:10.1517/13543784.16.8.1277. PMID 17685875.

### APPENDIX A

### Low Dose Naltrexone Adverse Effects

Even low dose Naltrexone has a high rate of adverse effects. In the study “Low Dose Naltrexone: Side Effects and Efficacy in Gastrointestinal Disorders” (Ploesser J, Weinstock L B, Thomas E, International Journal of Pharmaceutical Compounding; March 2010), the frequency of adverse effects of low dose Naltrexone in 206 patients was assessed retrospectively by a survey returned by 121 patients. Out of these patients 61/2% (74 patients) had side effects. No side effects were reported by only 38.8% (47 patients). And 32.4% (24 patients) had side effects, but were short-lived. In the group of patients who had side effects, Naltrexone treatment had to be terminated on 16.5% (20 patients) due to degree of adversity. The remaining patients were able to tolerate the side effects. The frequency of side effects for the group treated with 2.5 mg compared with the group treated with 5.0 mg daily differed in three symptoms, respectively:
Anxiety: (11.9% vs. 21.7%). Muscle pain: (4.5% vs. 15.2%). Diarrhea: (6.0% vs. 13.0%).
The side effects of Low Dose Naltrexone in 121 Patients, From the study “Low Dose Naltrexone: Side Effects and Efficacy in Gastrointestinal Disorders”

| | | Percentage of |
|---|---|---|
| | Adverse Event | Participants % |
| Neurological Side | Anxiety | 15.7 |
| Effects | Drowsiness | 11.6 |
| | Headache | 11.6 |
| | Dizziness | 10.7 |
| | Insomnia | 8.3 |
| | Muscle pain | 8.3 |
| | Vivid dreams | 5.0 |
| | Mood change | 3.3 |
| | Trouble concentrating | 1.7 |
| Gastrointestinal | Nausea | 12.4 |
| Side Effects | Abdominal pain | 11.6 |
| | Diarrhea | 8.3 |
| | Anorexia | 8.3 |
| Total side effects | | 61.2 |
| No side effects | | 38.8 |
| Terminated Naltrexone | | 16.5 |
| because of side effects | | |

The study shows that side effects of LDN occurred frequently (61%) and led to cessation of the treatment in 16.5% of the patients who returned the survey. The retrospective nature of the study and incomplete return of the surveys would likely bias the frequency to a higher number of adverse effects. There are only few studies that have evaluated side effects of LDN. In 42 IBS patients treated with ultra-LDN (0.5 mg), no side effects were reported. (Kariv R, Tiomny E, Grenshpon R et al. Low-dose Naltrexone for the treatment of irritable bowel syndrome: A pilot study. Dig Dis Sci 2006; 51(12): 2128-2133.).
End of Appendix A

That which is claimed is:

1. A composition for treatment of pain in a mammal comprising a synergistic ratio of (a) an opioid/TLR4 antagonist, or pharmaceutically acceptable salts or solvates thereof and (b) a direct-acting alpha-2 adrenergic agonist, or pharmaceutically acceptable salts or solvates thereof.

2. A composition comprising the formulation of claim 1, wherein the opioid/TLR4 antagonist is selected from a group consisting of naltrexone, norbinaltorphimine, nalmefene, naloxone, nalorphine, methylnaltrexone, samidorphan, cyprodime, naltrindole, amentoflavone, naltriben, norbinaltorphimine, 6-β-naltrexol, metabolites and pro drugs thereof, including all enantiomeric and epimeric forms as well as the appropriate mixtures thereof, or pharmaceutically acceptable salts or solvates of any thereof.

3. A composition comprising the formulation of claim 1, wherein, the opioid/TLR4 antagonist is naltrexone as well as pro drugs thereof or any enantiomeric and epimeric forms thereof, as well as the appropriate mixtures thereof, or pharmaceutically acceptable salts or solvates of any thereof.

4. A composition comprising the formulation of claim 3, wherein, the opioid/TLR4 antagonist is naltrexone in a sustained release formulation, as well as pro drugs thereof or any enantiomeric and epimeric forms thereof, as well as the appropriate mixtures thereof, or pharmaceutically acceptable salts or solvates of any thereof.

5. A composition comprising the formulation of claim 3, wherein, the opioid/TLR4 antagonist is (+)-naltrexone (dextro-naltrexone), as well as appropriate mixtures thereof, as well as pro drugs thereof, or pharmaceutically acceptable salts or solvates thereof.

6. A composition comprising the formulation of claim 1, wherein the direct-acting alpha 2 adrenergic agonist is selected from a group consisting of apraclonidine, brimonidine, clonidine, detomidine, dexmedetomidine, guanabenz, guanfacine, lofexidine, medetomidine, romifidine, tizanidine, tolonidine, xylazine and fadolmidine, or pharmaceutically acceptable salts or solvates of any thereof.

7. A composition comprising the formulation of claim 1, wherein the direct-acting alpha-2 adrenergic agonist is clonidine, or pharmaceutically acceptable salts or solvates thereof.

8. A composition comprising the formulation of claim 1, wherein the direct-acting alpha-2 adrenergic agonist is clonidine in a sustained release formulation, or pharmaceutically acceptable salts or solvates thereof.

9. A composition according to claim 1, wherein, the opioid/TLR4 antagonist is naltrexone, or pharmaceutically acceptable salts or solvates thereof, in a therapeutically effective amount and the direct-acting alpha-2 adrenergic agonist is clonidine, or pharmaceutically acceptable salts or solvates thereof, in therapeutically effective amount.

10. A composition according to claim 9, wherein naltrexone and clonidine, or pharmaceutically acceptable salts or solvates of any thereof, are in a weight to weight combination range which corresponds to a synergistic combination range of the order of 90:1 to 22.5:1 parts by weight.

11. A composition according to claim 10, wherein the dose range of naltrexone, or pharmaceutically acceptable salts or solvates thereof, is about 0.004 mg/kg-0.71 mg/kg. And wherein, the dose range of clonidine, or pharmaceutically acceptable salts or solvates thereof, is about 0.00018 mg/kg-0.0086 mg/kg per day.

12. A composition according to claim 10, wherein the human dose range of naltrexone is 0.25 mg-50 mg per day. And wherein, the human the dose range of clonidine is 0.0125 mg-0.6 mg, wherein said composition is formulated into a single fixed combination dosage form.

13. A composition according to claim 10, wherein the human dose range of naltrexone is 0.25 mg-15 mg per day. And wherein, the human the dose range of clonidine is 0.0125 mg-0.3 mg, wherein said composition is formulated into a single fixed combination dosage form.

14. A composition according to claim 10, wherein the composition is administered once, twice, three or four times through the day.

15. A composition of claim 10, wherein the therapeutically effective dose of the pharmaceutical composition is administered systemically, including but are not limited to mucosal, nasal, oral, parenteral, gastrointestinal, topical or sublingual routes.

16. A composition, according to claim 10, wherein said combination is in a single dosage form, and wherein, said single dosage form is in the form of tablets, lozenges, troches, hard candies, liquid, powders, sprays, creams, salves and suppositories.

17. A composition, according to claim 1 for treating, preventing and reversing pain.

18. A composition according to claim 1 for treating pain wherein said pain is back pain.

19. A composition according to claim 1 for treating pain wherein said pain is neuropathic pain.

20. A composition according to claim 1 for treating pain wherein said pain is migraine headache.

21. A composition according to claim 1 for treating pain wherein said pain is trigeminal neuralgia.

22. A composition according to claim 1 for treating pain wherein said pain is vulvodynia.

23. A composition according to claim 1 for treating pain wherein said pain is irritable bowel syndrome.

24. A composition according to claim 1 for treating pain wherein said pain is post herpetic neuralgia.

25. A composition according to claim 1 for treating pain wherein said pain is diabetic neuropathy.

26. A composition according to claim 1 for treating pain wherein said pain is nociceptive pain with an allodynic component.
